(19) United States
(12) Patent Application Publication
(10) Pub. No.: US 2024/0050638 A1
(43) Pub. Date: Feb. 15, 2024

(54) SURGICAL SUCTION TUBE INSTRUMENT WITH SEPARATED FINGER PADS

(71) Applicant: BR SURGICAL, LLC, Jacksonville, FL (US)

(72) Inventors: Timothy J. Reis, Jacksonville, FL (US); John Douglas Barrow, Jacksonville, FL (US)

(21) Appl. No.: 18/346,835

(22) Filed: Jul. 4, 2023

Related U.S. Application Data
(63) Continuation-in-part of application No. 17/819,392, filed on Aug. 12, 2022, now abandoned.

(51) Int. Cl. A61M 1/00; A61L 29/02

(52) U.S. Cl. CPC A61M 1/87; A61L 29/02; A61M 2205/586

(57) ABSTRACT

A disposable suction tube instrument includes a metal tubular tip with a curvature that facilitates operative site access, intimate surface contact while allowing space for aspiration of solids, liquids and gasses, without impeding access by other instruments. An integrally formed injection molded plastic handle includes upper and lower finger pads separated by gaps that provide finger recesses for gripping and also provide shrinkage relief to avoid distortion of the finger pads and of a teardrop control slot during post-molding cooling.

### RELATED APPLICATION

This invention is a continuation in part and claims the benefit of priority of U.S. Nonprovisional patent application Ser. No. 17/819,392, filed Aug. 12, 2022, the entire contents of which are incorporated herein and made a part hereof by this reference.

### FIELD OF THE INVENTION

This invention relates generally to surgical suction tubes, and, more particularly, to a disposable suction tube with an integrally formed injection molded handle having finger pads separated by a gap that mitigates deformations due to thermal gradients during molding.

### BACKGROUND OF THE INVENTION

A Fukushima suction tube is a suction device that surgeons (e.g., neurosurgeons) use to remove fluids and other materials from an operative site, in order to achieve an enhanced view of critical structures. The device includes a handle having a teardrop-shaped control slot in a finger pad that allows for precise regulation of suction. A channel extends through the handle. A suction hose attaches to one end of the handle. A cylindrical tubular tip is attached to the other end of the handle. The tip has an internal diameter measured on the French scale, typically ranging from 3F to 12F. The tip is malleable, allowing a practitioner (e.g., neurosurgeon) to bend the tip to provide good access to a surgical site.

Fukushima suction tubes are typically reusable devices. They are typically comprised of stainless steel. They must be cleaned and sterilized between procedures, after each use. Autoclave sterilization alone is insufficient, because, during use, contaminants enter and adhere to the interior of the tip. Manufacturers provide a thin flexible wire stylet to insert into the device and move back and forth for the purpose of mechanically removing any contained contaminants. This method of cleaning is not only tedious, but fraught with risk of missing contained contaminants.

During an operation, a surgeon uses an implement, such as a knife, to cut and penetrate or remove tissue, while a cautery device is used to cauterizes tissue, and the suction tube removes fluids and debris. The devices compete for access to the operative, with the surgeon's implement being of paramount importance. The malleable tubular tip of the suction devices is typically straight or slightly angled, neither of which allows access to the operative site without raising the handle to a position that blocks access by the knife or cautery. To gain access without substantially interfering with the surgeon's instrument, and avoid what medical professionals refer to as a sword fight, a practitioner will bend the malleable tip of the suction device. The bending is imprecise and may kink or partially occlude the tip. Sometimes the bending must be undone and redone several times to achieve the goal. As the inner diameter of the tip is small, any occlusion impedes flow and contributes to capture of contaminants. Such occlusion also compromises reusability. With the imprecisely bent tip, the practitioner may extend the tip into the operative sight, without positioning the handle in the way of the knife or cautery instrument.

Another shortcoming of conventional Fukushima suction tubes is limited ergonomics. The finger pads accommodate one finger (e.g., an index finger) on the bottom of the finger pad and a thumb on top of the finger pad. While other grips are possible, the device lacks defined finger placement structure, for stable gripping along the sides of the finger pad. The current devices are not well suited to gripping on each side between a thumb and middle finger while the index finger manages the teardrop-shaped control slot. Such a grip is particularly desirable for control and accurate positioning.

Lee (US 20080177251 A1) describes a conventional Fukushima suction tube with a straight tube, upper and lower plates extended from the middle section of the straight tube, a teardrop air control portion disposed at the middle of the upper plate and penetrating inside the straight tube. Space between Lee's upper and lower plates is occupied by a series of vertical ribs that extend between Lee's upper and lower plates. The ribs cause serious molding problems that preclude plastic injection molding.

When plastic is injected into a mold, the injected plastic contacts the mold. The mold is cooler than the plastic. At the point of contact, the plastic begins to cool via conduction. In areas where there are wider spaces, the greater mass of plastic stays molten longer. Thus, injected plastic cools at different rates based on mass/thickness. If a wall thickness suddenly changes, such as where a rib extends from a surface of Lee's plates, the thicker cross-section where the wall and rib meet will continue to cool and shrink long after the portions of the wall devoid of such ribs. This cooling differential causes a sink and warpage to form on the wall at the thicker area. Voids form when the solid skin is strong enough to withstand the negative pressure that builds as the polymer melt cools and shrinks without compensation. A sink is a dip (depression) in the surface of a plastic part that generally coincides with an internal structure. Warping occurs and sinks form because cooling is inversely related to mass. Each such defect (i.e., voids, sinks and warpage) would deform the shape of the plates and the shape of the teardrop slot, rendering the device unacceptable to users, e.g., neurosurgeons.

Additionally, conventional Fukushima suction tubes include finger placement surfaces that are generally planar, as in Lee (US 20080177251 A1). Conventional reusable devices are stainless steel structures. Fingers easily slide off the finger pads of such devices. On such surfaces, quickly finding the center of a pad, maintaining a finger centered and precisely controlling a teardrop slot can be extremely difficult.

A disposable injection molded Fukushima suction tube is needed. The tube should avoid deformations due to thermal gradients during injection molding. The tube should also facilitate accurate finger placement during use. The tube should also facilitate tip angling, without tip bending or occlusion, to facilitate flow during use.

The invention is directed to overcoming one or more of the problems and solving one or more of the needs as set forth above.

### SUMMARY OF THE INVENTION

To solve one or more of the problems set forth above, in an exemplary implementation of the invention, a disposable suction tube instrument includes a metal tubular tip with a curvature that facilitates operative site access, intimate surface contact while allowing space for aspiration of solids, liquids and gasses, without impeding access by other instruments. An integrally formed injection molded plastic handle includes upper and lower finger pads separated by a gap that provides finger recesses for gripping and also provides shrinkage relief to avoid distortion of a teardrop control slot during post-molding cooling.

An exemplary surgical suction tube device according to principles of the invention includes an integrally formed injection molded plastic handle and a tip. The handle includes a body with a longitudinal axis, a body width, an inlet end, an outlet end, an upper side, a lower side, the outlet end is opposite the inlet end, the upper side is opposite the lower side, a hose fitting at the outlet end, an inlet at the inlet end, and a central channel extending through the body from the outlet end to the inlet. The handle also includes a pair of finger pads, including an upper finger pad and a lower finger pad. The upper finger pad is disposed on the upper side of the body between the hose fitting and inlet. The lower finger pad is disposed on the lower side of the body between the hose fitting and the inlet and in alignment with the upper finger pad. Each finger pad includes a contact surface having a central axis, a pad width and a pad length. The central axis of each finger pad is parallel to the longitudinal axis of the body. The pad width is greater than the body width. The central axis of each finger pad and the longitudinal axis of the body is aligned. Side gaps are provided between the upper finger pad and lower finger pad, including a first side gap and a second side gap opposite the first side gap. A suction control slot extends through the upper finger pad to the central channel of the body of the handle. The tip is a metal tube with a free end and a proximal end opposite the free end. The proximal end of the tip is attached to the inlet of the body.

The first side gap includes a first height. The second side gap includes a second height. The upper finger pad has an upper finger pad thickness. The lower finger pad has a lower finger pad thickness. The first height is greater than each of the upper finger pad thickness and the lower finger pad thickness. The second height is about equal to the first height. The first height is effective to prevent shrinkage of the suction control slot during post-injection molding cooling and to provide a fingertip recess between the upper finger pad and the lower finger pad. The first height may be about equal to the sum of the upper finger pad thickness and the lower finger pad thickness. The first height may be about equal to 0.140 in. The first height may be at least as great as the sum of the upper finger pad thickness and the lower finger pad thickness. The pad length (i.e., length of each finger pad) may be greater than the pad width. The pad length is greater than the length of the suction control slot. The pad width is greater than a maximum width of the suction control slot 0.140

The metal tube comprising the tip is a stainless-steel tube with an inner diameter from 3F to 12F. The free end of the tip is blunted, deburred and buffed. The metal tube may be curved and have a radius of curvature of 4 to 12 inches and extend for an arc angle of 40 to 60 degrees; and/or have a radius of curvature of 4 to 12 inches, and a chord measured from the free end to the proximal end having a length of 3.5 to 7.0 inches; and/or have an arc angle of 40 to 60 degrees, and a chord measured from the free end to the proximal end having a length of 3.5 to 7.0 inches.

In an embodiment, a surgical suction tube device includes a tip and an integrally formed injection molded plastic handle. The handle is an injection molded plastic structure. The handle includes a body with a longitudinal axis, a body width, an inlet end, an outlet end, an upper side, and a lower side. The outlet end is opposite the inlet end. The upper side is opposite the lower side. A hose fitting is provided at the outlet end. An inlet is provided at the inlet end. A central channel extends through the body from the outlet end to the inlet. A pair of finger pads, including an upper finger pad and a lower finger pad, is provided. The upper finger pad is disposed on the upper side of the body between the hose fitting and inlet. The lower finger pad is disposed on the lower side of the body between the hose fitting and the inlet and in alignment with the upper finger pad. Each finger pad includes a concave contact surface having a central axis, a pad width and a pad length. The central axis of each finger pad is parallel to the longitudinal axis of the body. The pad width is greater than the body width. The first portion of each finger pad extends beyond a first side of the body. A second portion of each finger pad extends beyond a second side of the body. Side gaps are provided between the upper finger pad and lower finger pad. The gaps include a first side gap and a second side gap opposite the first side gap. The first side gap is coextensive with the first portion of each finger pad extending beyond a first side of the body. The second side gap is coextensive with the second portion of each finger pad extending beyond a second side of the body. The first side gap is devoid of any structure extending between the upper finger pad and lower finger pad. The second side gap is devoid of any structure extending between the upper finger pad and lower finger pad. The side gaps provide shrinkage relief during molding.

A suction control slot extends through the upper finger pad to the central channel of the body of the handle. The tip includes a metal tube with a free end and a proximal end opposite the free end. The proximal end of the tip is attached to the inlet of the body.

In one embodiment, the first side gap has a first height and the second side gap has a second height. The upper finger pad has an upper finger pad thickness. The lower finger pad has a lower finger pad thickness. The first height is greater than each of the upper finger pad thickness and the lower finger pad thickness, to provide shrinkage relief during molding. The first height is effective to prevent shrinkage of the suction control slot during post-injection molding cooling. The first height is also effective to provide a fingertip recess between the upper finger pad and the lower finger pad. The first height is equal to the sum of the upper finger pad thickness and the lower finger pad thickness. In one embodiment, the first height is about 0.140 in. The first height is at least as great as the sum of the upper finger pad thickness and the lower finger pad thickness. The pad length is greater than the pad width. The pad length is greater than the length of the suction control slot. The pad width is greater than a maximum width of the suction control slot. The central axis of each finger pad and the longitudinal axis of the body is aligned.

The metal tube may be a stainless-steel tube with an inner diameter from 3F to 12F. The metal tube may have a free end that is blunted, deburred and buffed. The metal tube may be curved and have a radius of curvature of 4 to 12 inches and extend for an arc angle of 40 to 60 degrees and/or define a chord measured from the free end to the proximal end having a length of 3.5 to 7.0 inches.

### BRIEF DESCRIPTION OF THE DRAWINGS

The foregoing and other aspects, objects, features and advantages of the invention will become better understood with reference to the following description, appended claims, and accompanying drawings, where:

FIG. 1 is a profile view of an exemplary suction tube device according to principles of the invention.

FIG. 2 is a perspective view of an exemplary suction tube device according to principles of the invention.

FIG. 3 is another perspective view of an exemplary suction tube device according to principles of the invention.

FIG. 4 is a plan view of a handle of an exemplary suction tube device according to principles of the invention.

FIG. 5 is a profile view of a handle of an exemplary suction tube device according to principles of the invention.

FIG. 6 is a bottom view of a handle of an exemplary suction tube device according to principles of the invention.

FIG. 7 is a perspective view of a handle of an exemplary suction tube device according to principles of the invention.

FIG. 8 is another perspective view of a handle of an exemplary suction tube device according to principles of the invention.

FIG. 9 is a section view (A-A) of a handle of an exemplary suction tube device according to principles of the invention.

FIG. 10 is a section view (B-B) of a handle of an exemplary suction tube device according to principles of the invention.

FIG. 11 is a perspective view of a section (B-B) of a handle of an exemplary suction tube device according to principles of the invention.

FIG. 12 is a specification for a curved tubular tip of an exemplary suction tube device according to principles of the invention.

FIG. 13 is another specification for a curved tubular tip of an exemplary suction tube device according to principles of the invention.

FIG. 14 is another specification for a curved tubular tip of an exemplary suction tube device according to principles of the invention.

FIG. 15 is another specification for a curved tubular tip of an exemplary suction tube device according to principles of the invention.

FIG. 16 provides a profile view of an exemplary suction tube device with a tip at an operative site according to principles of the invention.

FIG. 17 is a dimensioned plan view of a handle of an exemplary suction tube device according to principles of the invention.

FIG. 18 is a dimensioned side view of a handle of an exemplary suction tube device according to principles of the invention.

FIG. 19 is a dimensioned front view of a handle of an exemplary suction tube device according to principles of the invention.

FIG. 20 conceptually illustrates problems that arise due to thermal gradients attributed to structures such as ribs between finger pads.

Those skilled in the art will appreciate that the figures are not intended to be drawn to any particular scale; nor are the figures intended to illustrate every embodiment of the invention. The invention is not limited to the exemplary embodiments depicted in the figures or the specific components, configurations, shapes, relative sizes, ornamental aspects or proportions as shown in the figures.

### DETAILED DESCRIPTION

An exemplary suction tube device according to principles of the invention includes a tubular tip with a curvature that facilitates operative site access, intimate surface contact while allowing space for aspiration of solids, liquids and gasses, without impeding access by other instruments. The device is disposable, which avoids risks of contamination. The handle is an integrally formed (integrally-formed) structure, which is comprised of injection molded plastic. The handle includes upper and lower finger pads separated by a functionally-significant gap. The gap provides finger recesses, such as for gripping on each side between a thumb and middle finger while the index finger manages a teardrop-shaped control slot. Such a grip is particularly desirable for control and accurate positioning. The gap also provides shrinkage relief. Without the gap, after molding, the teardrop-shaped control slot would shrink, thereby limiting and distorting flow control. In sum, the device is easy to grip including along its sides, disposable, easy to position at an operative site without impeding visibility or access by other instruments, and provides an industry-accepted teardrop-shaped control slot.

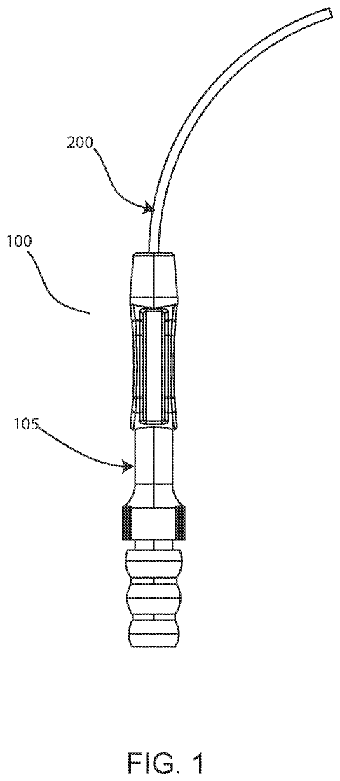

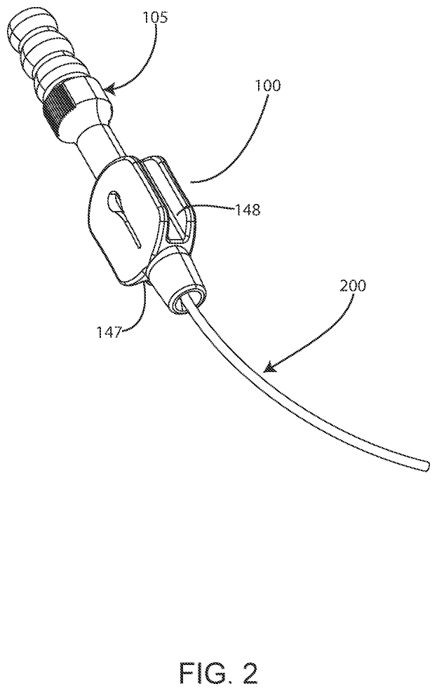

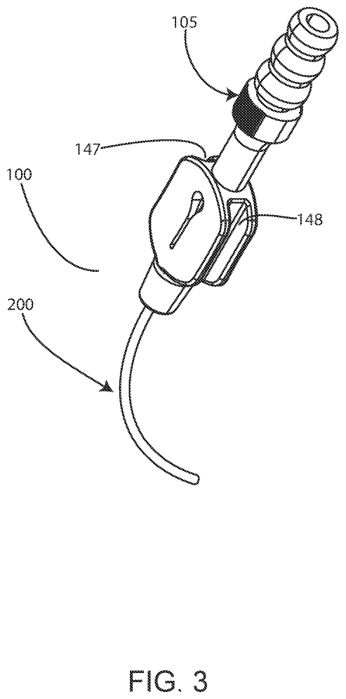

Referring to FIGS. 1-3, components of an exemplary suction tube device 100 according to principles of the invention include a handle 105 and a curved tip 200. The curved tip 200 attaches to the handle 105. The tip 200 is metal tube. The handle 105 is a molded plastic structure. Each component is described in greater detail below.

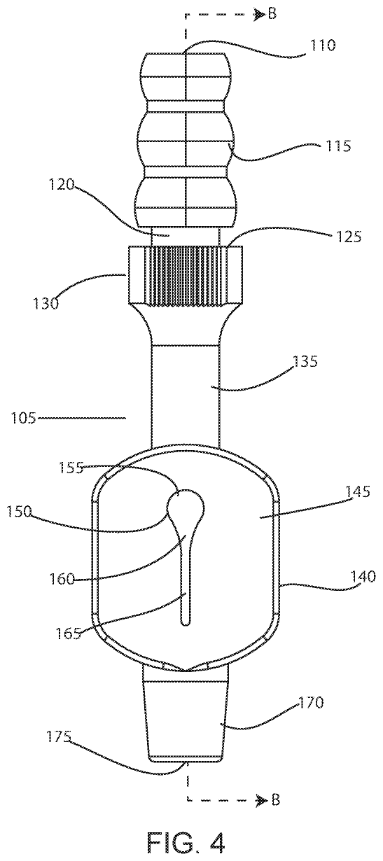

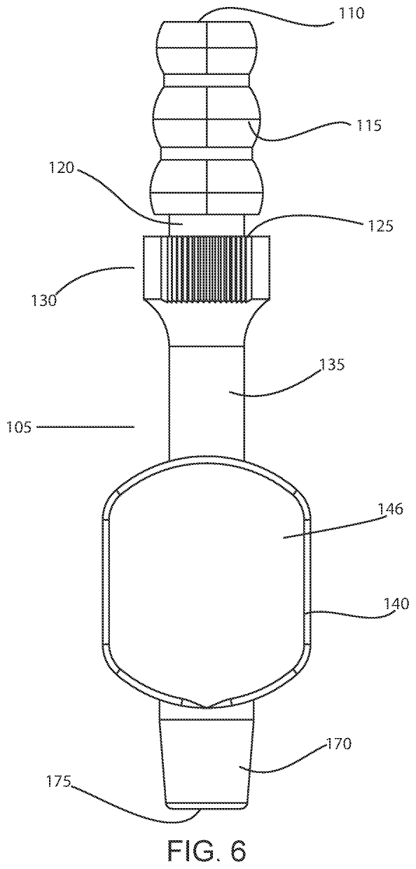

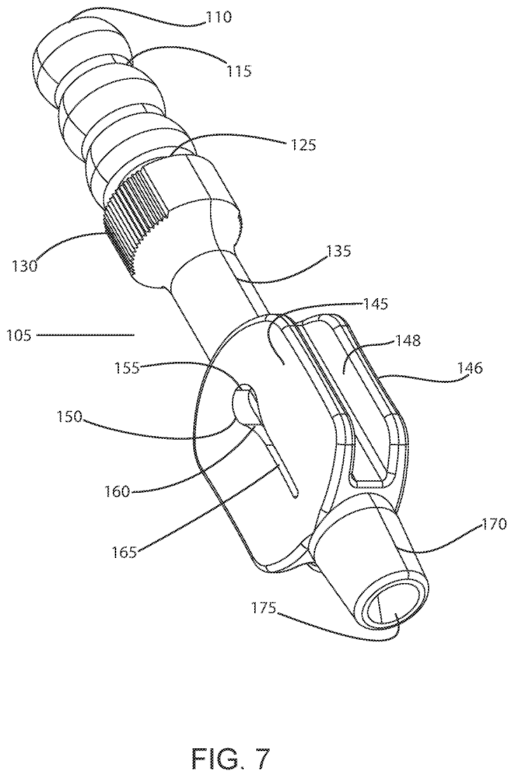

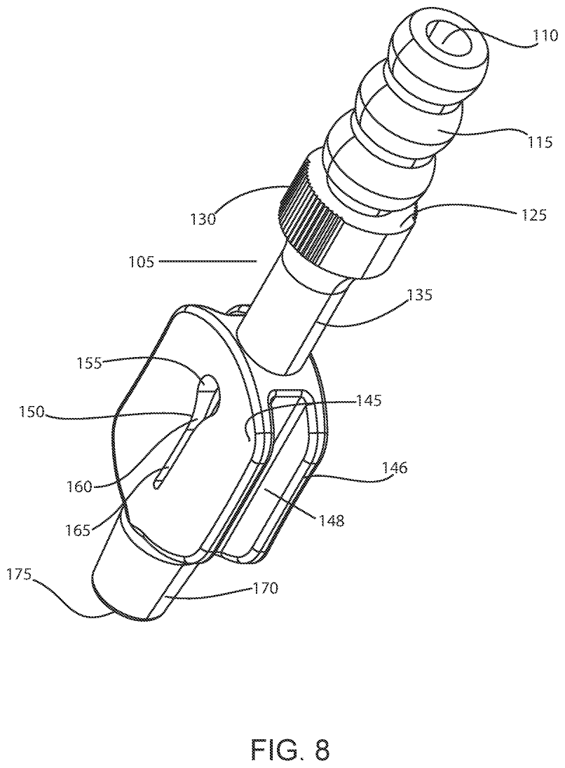

FIGS. 4-8 provide various views of the handle 105, without the tip 200. The proximal end of the handle (i.e., the end of the handle to which a suction hose attaches) includes a hose fitting 115 with smooth spherical barbs. The maximum outer diameter of the barbs is slightly greater than the inner diameter of the suction hose. The hose, being a resilient elastomer, stretches to receive the fitting 115. The barbs omit any sharp edges that may damage the interior of a suction tube. The outlet 110 is an opening in the distal end through which aspirated matter flows. A neck 120 connects the fitting 115 to a collar 130. The collar includes a shoulder 125, which is a distal edge, having a width (e.g., outer diameter) that is greater than the maximum outer diameter of the barbs of the fitting 115. The shoulder 125 defines a stopping point for a hose. A hose cannot be advanced over the fitting 115 beyond the shoulder 125. The collar 130 includes a textured surface (e.g., fluted or ribbed) to facilitate gripping. To facilitate hose installation, the collar may be repeatedly rotated in one direction and then an opposite direction, between a user's fingers, to facilitate advancing a hose onto the fitting 115.

A body 135 extends from the collar 130 to the tip joint 170. The body 135 is a tubular structure. As discussed below, a central channel extends through the body. Aspirated matter flows through the central channel of the body 135.

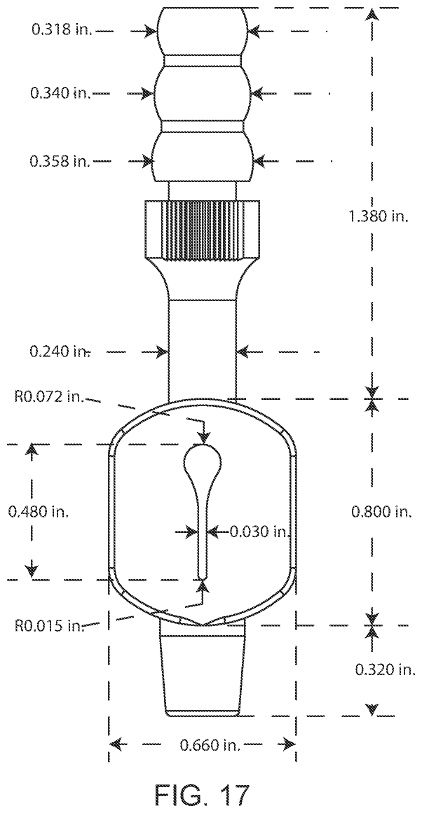

A finger grip 140 includes an upper finger pad 145 and a lower finger pad 146. Each finger pad 145, 146 is a broad thin panel that provides a surface to receive at least one fingertip. In the nonlimiting example of FIG. 17, each finger pad has a length of about 0.800 in. and a width of about 0.660 in., which are greater than the length (0.480) and widths (0.03 in. and 0.142 in.) of the suction control slot.

In the exemplary embodiment shown in FIGS. 4-8, each finger pad 145, 146 has a badge shape, with parallel sides, a curved proximal end, and a slightly pointed distal end. However, the invention is not limited to finger pads having a badge shape. Other shapes, including but not limited to rectangular, oval and circular, may be used without departing from the scope of the invention.

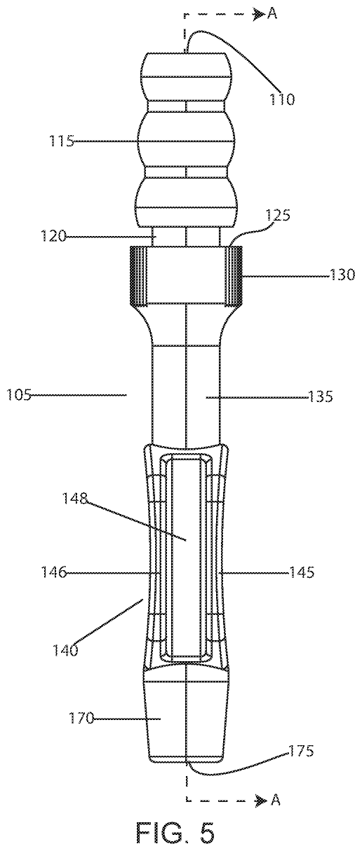

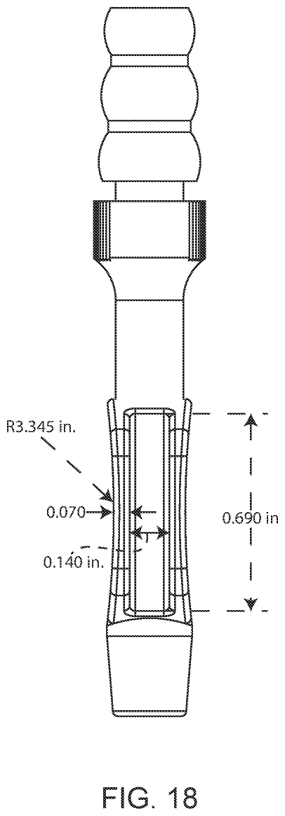

As evident in the side view of FIG. 5, each finger pad 145, 146 is slightly concave. The concavity provides a surface for continuous finger contact. In the nonlimiting example of FIG. 18, the concave surface has a radius of curvature of about 3.345 in. Concave surfaces having radii of curvature of 2.5 to 4.5 inches are preferred. Such radii accommodate a wide range of adult fingers, providing a tactile concavity that facilitates centering. An appreciably larger radius compromises tactile perception. An appreciably smaller radii constrains a finger and may actually prevent sealing contact over the slot 150.

Additionally, the edges of the finger pads 145, 146 are filleted (i.e., rounded) to avoid any sharp corners that may irritate fingers. The concavity is functionally significant, as it greatly facilitates proper finger positioning. Finger positioning is critical to efficient and optimal use of the device during surgery.

A slot-like gap 147, 148 is provided between the finger pads 145, 146, along each side of the handle 105. Each gap 147, 148 is devoid of structure between the finger pads 145, 146. The handle 105 is symmetrical. The gap 147, 148 extends, on each side of the handle 105, between the outwardly extending portion of each finger pad 145, 146, to the body 135 of the handle 105.

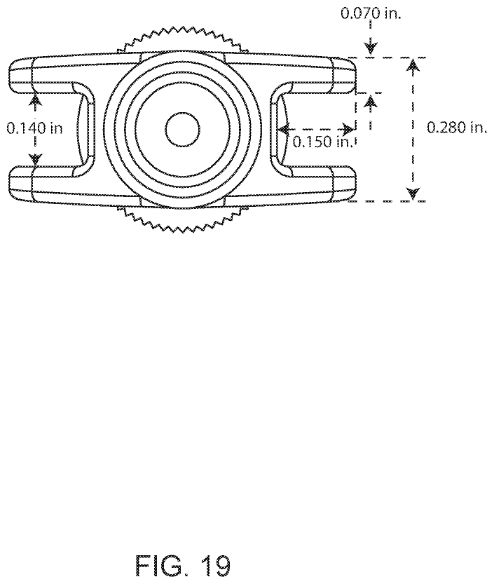

The configuration and arrangement of the gaps 147, 148, and finger pads 145, 146, including their relative dimensions and proportions are important to facilitate shrinkage relief during molding. The height of the gap 147, 148 is greater than the thickness of each finger pad 145, 146. In the nonlimiting example of FIGS. 17-19, the thickness of each finger pad is about 0.070 in. The thickness may vary along the width of the finger pad. In the dimensioned example, the height of the gap is about 0.140 in., which is about twice the thickness of a finger pad, or about equal to the sum of the thicknesses of the upper and lower finger pads. In the nonlimiting example of FIG. 19, the depth of the gap is about 0.150 in., which is nearly equal to, slightly larger than the gap height, and which is about (i.e., slightly greater than) twice the thickness of a finger pad, or about (i.e., slightly greater than) equal to the sum of the thicknesses of the upper and lower finger pads.

The gap 147, 148 is functionally significant. It provides a recess for fingers. With the gaps 147, 148, a user may grip the handle 105 with a thumb in the gap on one side of the finger pads 145, 146 and a middle finger in the gap on the other side of the finger pads 145, 146, while the index finger manages the teardrop-shaped control slot 150. Such a grip is particularly desirable for control and accurate positioning. It is an alternative to the conventional grip, in which a thumb engages one finger pad surface, while the index or middle finger engages the other finger pad surface.

The gap 147, 148, being devoid of structure between the finger pads 145, 146, provides shrinkage relief If the gap 147, 148 was omitted and the sides between the finger pads 145, 146 were solid, as in a conventional Fukushima suction tube, or if the gap 147, 148 contained structures extending between finger pads 145, 146, the finger pads 145, 146 would warp and the teardrop-shaped control slot 150 would deform during cooling, after injection molding. Shrinkage of the injection molded handle 105 during post-molding cooling would appreciably distort the shape and dimensions of the pads 145, 146 and teardrop-shaped control slot 150 in the upper finger pad 145. Such distortion would compromise the utility, and practitioner acceptance, of the handle 105. The gaps 147, 148 allow post-molding cooling of the thin separated finger pads 145, 146 without appreciable distortion of the teardrop-shaped control slot 150.

When plastic is injected into a mold, the injected plastic contacts the mold. The mold is cooler than the plastic. At the point of contact, the plastic begins to cool via conduction. In areas where there are wider spaces, the greater mass of plastic stays molten longer. Thus, injected plastic cools at different rates based on mass/thickness.

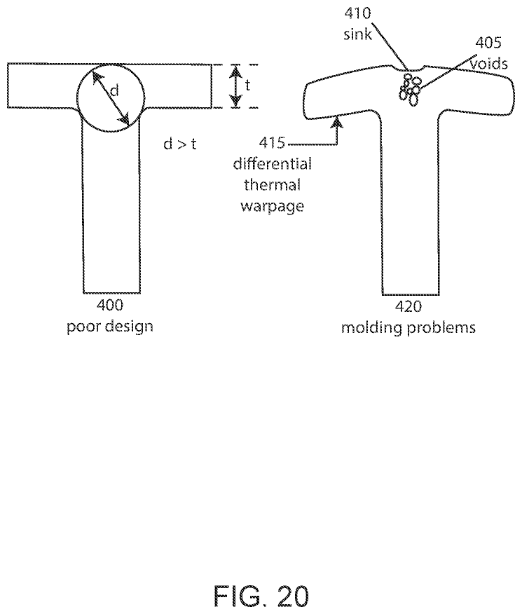

If a wall thickness suddenly changes, such as where a rib extends from the back side of a wall, the thicker cross-section where the wall and rib meet will continue to cool and shrink long after the portions of the wall devoid of such ribs. This cooling differential causes a sink and warpage to form on the wall at the thicker area. Depending on the mass differential, the sink could be fairly minor or it could produce a void. Voids form when the solid skin is strong enough to withstand the negative pressure that builds as the polymer melt cools and shrinks without compensation. The sink is a dip (depression) in the surface of a plastic part that generally coincides with an internal structure. Warping occurs and sinks form because cooling is inversely related to mass. Thus, thicker plastic cools slower than thinner plastic. Thicker plastic retains heat longer than thinner plastic. See, Plastic Part Design for Injection Molding: An Introduction, 2nd Edition, by Robert A. Malloy, Hanser Publishers (2010), ISBN: 978-1-56990-436-7, 1-56990-436-7, p. 67; Complete Part Design Handbook For Injection Molding of Thermoplastics, by E. Alfredo Campo, Hanser Publishers (2006), ISBN: 978-3-446-40309-3,3-446-40309-4, pp. 211-214. The schematic provided as FIG. 20 conceptually illustrates the problem of a poor design 400.

Voids 405, sinks 410, and warpage 415 are defects that compromise utility of the device. First, voids 405, sinks 410, and warpage 415 compromise structural integrity of the upper and lower plates. Concomitantly, the defects would change the shape (i.e., contour) of the upper and lower pads 145, 146, by adding undulating warpage and sinks. Such defects would interfere with a finger maintaining a seal over portions of the air control slot 150. Neurosurgeons, who are, by necessity, very precise, would shun any flow controller that includes deformed upper or lower plates.

Additionally, such defects would deform the shape of the teardrop-shaped slot 150. The shape and size of the slot 150 is consistent among such surgical suction devices. A neurosurgeon could not tolerate any variation from the standard shape and size, as it would interfere with proper suction. Neurosurgeons expect a certain shape and size. Through experience, they know exactly how much to move their finger to increase, decrease, disrupt or maximize suction. A distorted slot would prevent such use, with potentially serious consequences.

During molding, the gaps 147, 148, are undercuts. Mold lifters (or sliders) eject the handle upon injection molding. The lifters move in two dimensions to release and facilitate ejection. The invention is not limited to any particular lifters or sliders for part release and ejection.

The suction control slot 150 extends through the upper finger pad 145 to the central channel of the handle 105. The exemplary handle 105 depicted in FIGS. 4-8 features a teardrop shaped suction control slot, which allows for extremely precise regulation of suction. Such a slot shape is a well-known feature of Fukushima suction tubes. It is a feature with which surgeons have great familiarity. It is a control slot shape that has achieved widespread acceptance in the surgical community. The teardrop shaped slot 150 includes a bulbous proximal end 155, a narrow slit 165 with parallel sides 165, and a funnel-shaped transition 160 that joins the bulbous proximal end 155 to the narrow slit 165. Significantly, an injection molded handle 105 according to principles of the invention features a teardrop shaped control slot 150. The shape, proportions and the dimensions of the slot 150, which are critical for practitioner use and acceptance, are maintained after the injection molded handle 105 has cooled, because the gaps 147, 148 provide shrinkage relief.

A tip joint 170 includes an inlet 175 into which a tip 200 is mounted. The tip 200 is coupled to the tip joint 170 via bonding, such as with a glue. The glue is suitable for joining the stainless-steel tip 200 to the plastic of the handle 105.

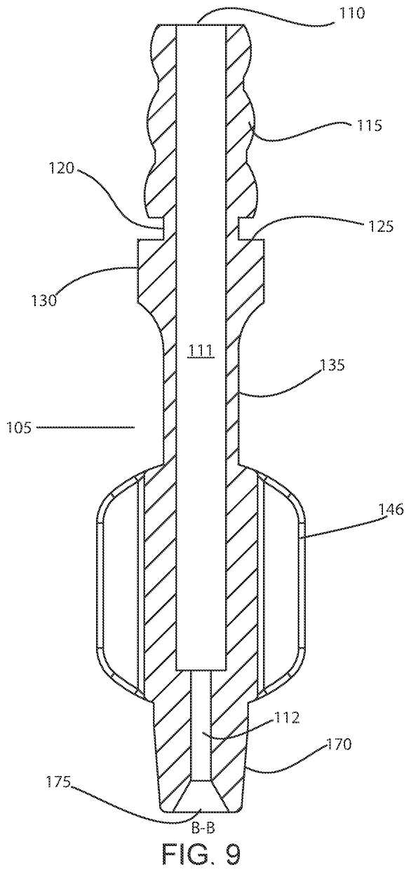

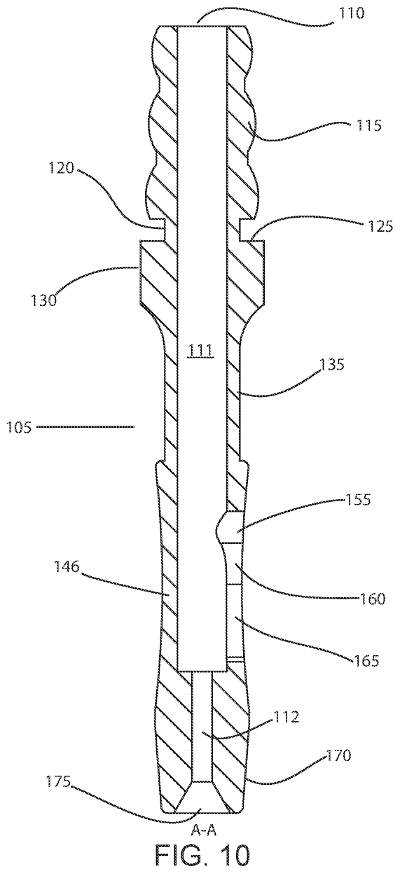

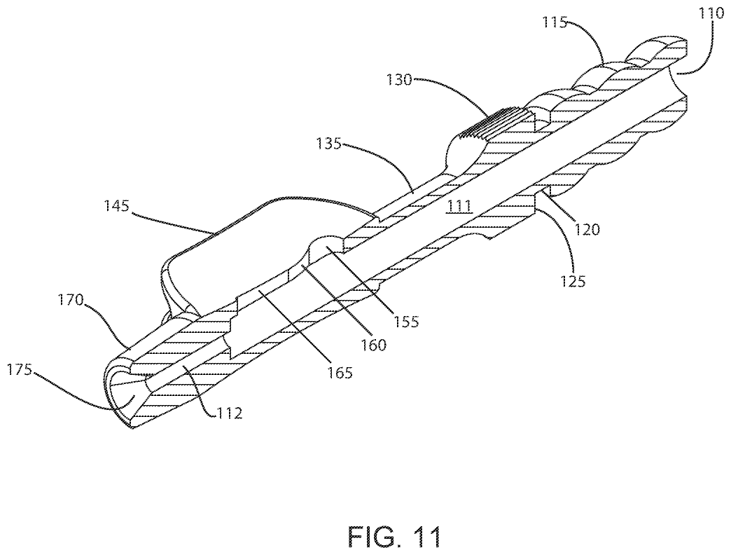

Referring to the section views of FIGS. 9-11, a primary central channel 111 extends from the outlet 110 to the finger grip 140. The primary central channel 111 has a circular cross section. A secondary central channel 112 extends from the primary central channel 111 to the inlet 175. The secondary central channel 112 has a circular cross section. The secondary central channel 112 has a smaller diameter than that of the primary central channel 111. The diameter of the primary central channel 111 may be about equal to the unstretched inner diameter of the suction hose that connects to the fitting 115. The diameter of the secondary central channel 112 may be about equal to the outer diameter of the tip 200. The inlet 175 is funnel (i.e., frusto-conical) shaped. The funnel shape allows sufficient adhesive thickness for a range of tip diameters. Effective bonding requires sufficient adhesive, e.g., a 0.1 to 0.5 mm thick layer, between the substrates being bonded.

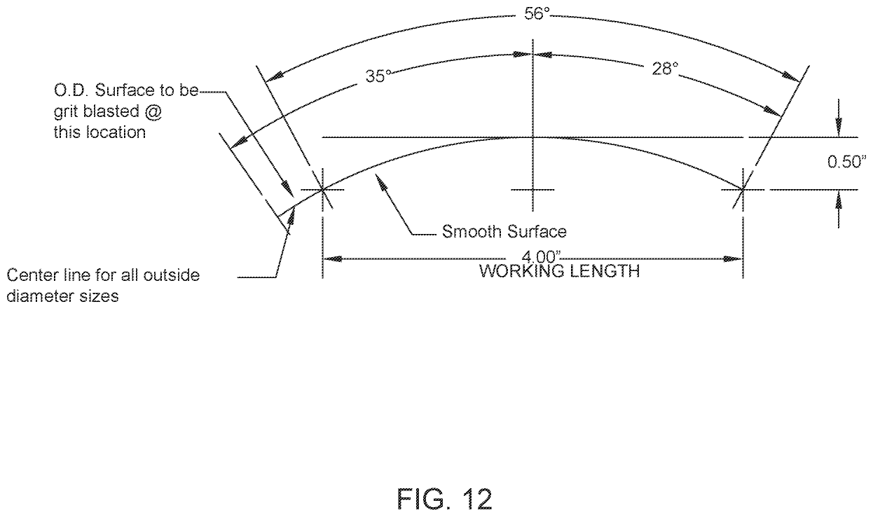

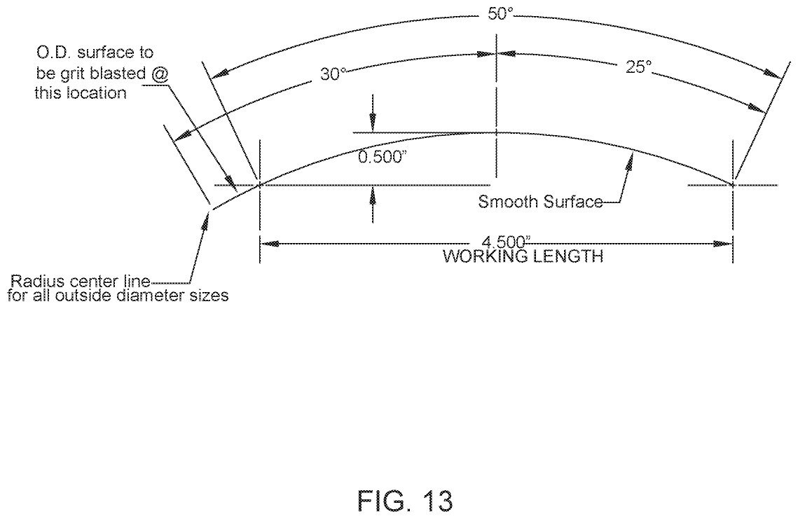

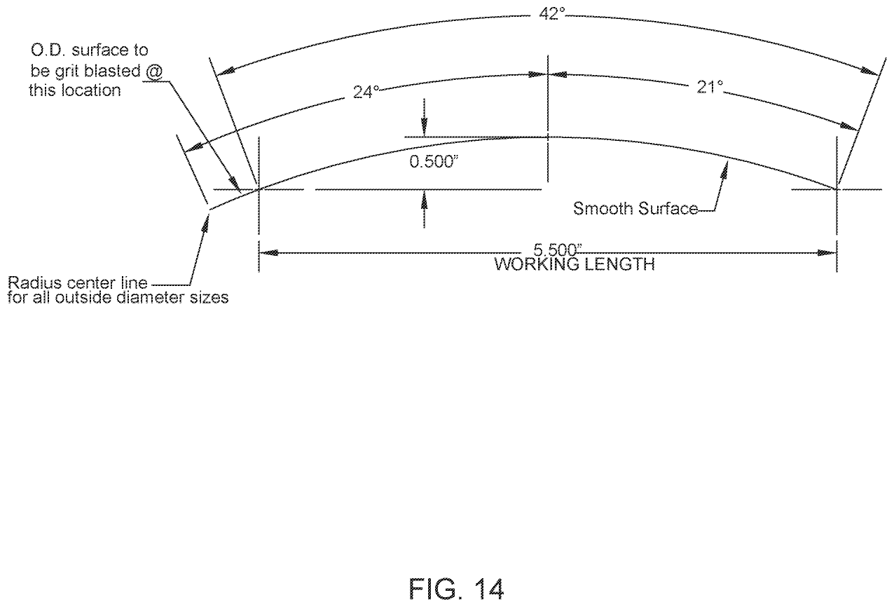

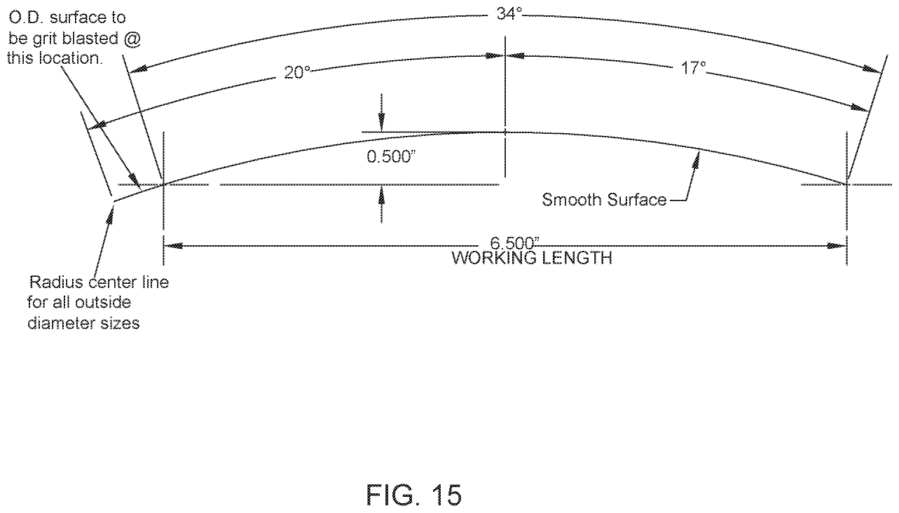

FIGS. 12-15 provide non-limiting examples of specifications (dimensions) for a curved tubular tip 200 of an exemplary suction tube device according to principles of the invention. The tip 200 is a stainless-steel tube. By way of example, the tip 200 may be comprised of SAE 304 or 316L stainless steel. The inner diameter of the tip 200 is measured on the French scale and may range from 3F to 12F. One end of the tip 200 is received in the inlet 175. The other end of the tip 200 is a free end, which accesses the operative site. The free end of the tip 200 may be blunted (e.g., filleted), deburred and buffed to provide an atraumatic structure, thereby reducing risk of tissue damage or other injury.

A radius of curvature may be computed using the chord length and arc angle in FIGS. 12-15, via the following equation, in which l is the chord length, θ is the arc angle and r is the radius of curvature:

$$r = \frac{l}{2 \cdot sin ( \frac{θ}{2} )}$$

The radius of curvature for each example is provided in the table below. For chord lengths of about 4 to 6.5 inches (+/−0.25 inches), these radii, radii in between these radii, and radii within an inch of the minimum and maximum radii in the table, are effective for providing a curvature of the tip that facilitates access in accordance with the principles of the invention.

| l (inches) | θ (deg.) | r (inches) |
|---|---|---|
| 4 | 56 | 4.260 |
| 4.5 | 50 | 5.324 |
| 5.5 | 42 | 7.674 |
| 6.5 | 34 | 11.116 |

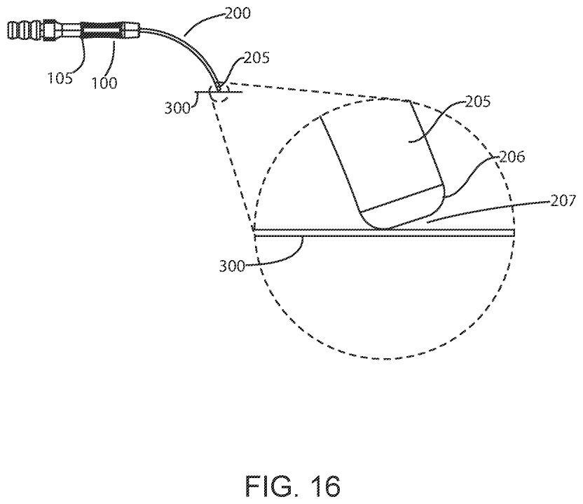

FIG. 16 provides a profile view of an exemplary suction tube device 100 with a tip 200 at an operative site according to principles of the invention. The free end 205 of the tip 200 includes smoothed or rounded (e.g., filleted) edges 206 to avoid causing trauma. During use, the free end 205 may be maintained at an angle relative to the contacted surface 300 of the operative site. The angle creates a gap 208 between the inlet opening 207 in the free end 205 and the contacted surface 300. Without the angle, the gap may be eliminated. Without the gap, efficiency of the tip 200 is compromised. Solid, liquid and gaseous matter may enter the inlet opening 207 in the free end 205 through the gap 208. Concomitantly, due to the curve and length of the tip, the handle 105 is somewhat removed from the operative site. Thus the suction tube device 100 may be used without interfering with other instruments (e.g. cautery and knife) that may require access to the operative site at the same time.

Any dimensions, amounts or concentrations are provided as approximations for a particular embodiment, unless expressly stated otherwise. The dimensioned views of a handle of an exemplary suction tube device according to principles of the invention, as provided in FIGS. 17-19, are nonlimiting examples. The dimensions may be varied without compromising utility and without departing from the scope of the invention. Varied dimensions, amounts or concentrations that do not substantially impair utility of the invention come within the spirit and scope of the invention. Subject to the foregoing, unless otherwise specified herein, dimensions may be varied by ±5% without departing from the scope of the invention. Thus, except as otherwise stated herein, about equal to means equal to or within ±5% of being equal. Similarly, at least about means as great as or within ±5% of being as great as.

While an exemplary embodiment of the invention has been described, it should be apparent that modifications and variations thereto are possible, all of which fall within the true spirit and scope of the invention. With respect to the above description then, it is to be realized that the optimum relationships for the components and steps of the invention, including variations in order, form, content, function and manner of operation, are deemed readily apparent and obvious to one skilled in the art, and all equivalent relationships to those illustrated in the drawings and described in the specification are intended to be encompassed by the present invention. The above description and drawings are illustrative of modifications that can be made without departing from the present invention, the scope of which is to be limited only by the following claims. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents are intended to fall within the scope of the invention as claimed.

What is claimed is:

1. A surgical suction tube device comprising a tip and an integrally formed injection molded plastic handle, the handle comprising an injection molded plastic structure, including:
a body with a longitudinal axis, a body width, an inlet end, an outlet end, an upper side, a lower side, the outlet end being opposite the inlet end, the upper side being opposite the lower side, a hose fitting at the outlet end, an inlet at the inlet end, and a central channel extending through the body from the outlet end to the inlet;
a pair of finger pads, including an upper finger pad and a lower finger pad, the upper finger pad being disposed on the upper side of the body between the hose fitting and inlet, the lower finger pad being disposed on the lower side of the body between the hose fitting and the inlet and in alignment with the upper finger pad, each finger pad of the pair of finger pads comprising a concave contact surface having a central axis, a pad width and a pad length, the central axis of each finger pad being parallel to the longitudinal axis of the body, and the pad width being greater than the body width, and a first portion of each finger pad extending beyond a first side of the body, and a second portion of each finger pad extending beyond a second side of the body;
side gaps between the upper finger pad and lower finger pad, including a first side gap and a second side gap opposite the first side gap, the first side gap being coextensive with the first portion of each finger pad extending beyond a first side of the body, and the second side gap being coextensive with the second portion of each finger pad extending beyond a second side of the body, and the first side gap being devoid of any structure extending between the upper finger pad and lower finger pad, and the second side gap being devoid of any structure extending between the upper finger pad and lower finger pad, wherein the side gaps provide shrinkage relief; and
a suction control slot extending through the upper finger pad to the central channel of the body of the handle; and
the tip comprising a metal tube with a free end and a proximal end opposite the free end, the proximal end of the tip being attached to the inlet of the body.

2. The surgical suction tube device of claim 1, the first side gap including a first height, the second side gap including a second height, the upper finger pad having an upper finger pad thickness, and the lower finger pad having a lower finger pad thickness, the first height being greater than each of the upper finger pad thickness and the lower finger pad thickness, thereby providing shrinkage relief.

3. The surgical suction tube device of claim 1, each concave contact surface having a radius of curvature of 2.5 to 4.5 inches.

4. The surgical suction tube device of claim 2, the first height being effective to provide a fingertip recess between the upper finger pad and the lower finger pad.

5. The surgical suction tube device of claim 2, the first height being equal to the sum of the upper finger pad thickness and the lower finger pad thickness.

6. The surgical suction tube device of claim 5, the first height being 0.140 in.

7. The surgical suction tube device of claim 2, the first height being at least as great as the sum of the upper finger pad thickness and the lower finger pad thickness.

8. The surgical suction tube device of claim 7, the first height being 0.140 in.

9. The surgical suction tube device of claim 1, the pad length being greater than the pad width.

10. The surgical suction tube device of claim 1, the pad length being greater than the length of the suction control slot.

11. The surgical suction tube device of claim 1, the pad width being greater than a maximum width of the suction control slot.

12. The surgical suction tube device of claim 1, the central axis of each finger pad and the longitudinal axis of the body being aligned.

13. The surgical suction tube device of claim 1, the metal tube comprising a stainless-steel tube with an inner diameter from 3F to 12F.

14. The surgical suction tube device of claim 13, the metal tube including a free end, the free end being blunted, deburred and buffed.

15. The surgical suction tube device of claim 13, the metal tube being curved and having a radius of curvature of 4 to 12 inches and extending for an arc angle of 40 to 60 degrees.

16. The surgical suction tube device of claim 13, the metal tube being curved and having a radius of curvature of 4 to 12 inches, and a chord measured from the free end to the proximal end having a length of 3.5 to 7.0 inches.

17. The surgical suction tube device of claim 13, the metal tube being curved and extending for an arc angle of 40 to 60 degrees, and a chord measured from the free end to the proximal end having a length of 3.5 to 7.0 inches.

18. An integrally formed injection molded plastic surgical suction tube handle, comprising a body with a longitudinal axis, a body width, an inlet end, an outlet end, an upper side, a lower side, the outlet end being opposite the inlet end, the upper side being opposite the lower side, a hose fitting at the outlet end, an inlet at the inlet end, and a central channel extending through the body from the outlet end to the inlet;
a pair of finger pads, including an upper finger pad and a lower finger pad, the upper finger pad being disposed on the upper side of the body between the hose fitting and inlet, the lower finger pad being disposed on the lower side of the body between the hose fitting and the inlet and in alignment with the upper finger pad, each finger pad of the pair of finger pads comprising a contact surface having a central axis, a pad width and a pad length, the central axis of each finger pad being parallel to the longitudinal axis of the body, and the pad width being greater than the body width, and a first portion of each finger pad extending beyond a first side of the body, and a second portion of each finger pad extending beyond a second side of the body;
side gaps between the upper finger pad and lower finger pad, including a first side gap and a second side gap opposite the first side gap, the first side gap being coextensive with the first portion of each finger pad extending beyond a first side of the body, and the second side gap being coextensive with the first portion of each finger pad extending beyond a first side of the body, and the first side gap being devoid of any structure, and the second side gap being devoid of any structure;
a suction control slot extending through the upper finger pad to the central channel of the body of the handle;
the handle being comprised of injection molded plastic.

19. The integrally formed injection molded plastic surgical suction tube handle of claim 18, the first side gap including a first height, the second side gap including a second height, the upper finger pad having an upper finger pad thickness, and the lower finger pad having a lower finger pad thickness, the first height being greater than each of the upper finger pad thickness and the lower finger pad thickness, the first height being effective to prevent shrinkage of the suction control slot during post-injection molding cooling, and the first height being effective to provide a fingertip recess between the upper finger pad and the lower finger pad.

20. A surgical suction tube device comprising a tip and an integrally formed injection molded plastic handle:
the handle comprising:
a body with a longitudinal axis, a body width, an inlet end, an outlet end, an upper side, a lower side, the outlet end being opposite the inlet end, the upper side being opposite the lower side, a hose fitting at the outlet end, an inlet at the inlet end, and a central channel extending through the body from the outlet end to the inlet;
a pair of finger pads, including an upper finger pad and a lower finger pad, the upper finger pad being disposed on the upper side of the body between the hose fitting and inlet, the lower finger pad being disposed on the lower side of the body between the hose fitting and the inlet and in alignment with the upper finger pad, each finger pad of the pair of finger pads comprising a contact surface having a central axis, a pad width and a pad length, the central axis of each finger pad being parallel to the longitudinal axis of the body, and the pad width being greater than the body width;
side gaps between the upper finger pad and lower finger pad, including a first side gap and a second side gap opposite the first side gap, the first side gap being coextensive with the first portion of each finger pad extending beyond a first side of the body, and the second side gap being coextensive with the first portion of each finger pad extending beyond a first side of the body, and the first side gap being devoid of any structure, and the second side gap being devoid of any structure;
a suction control slot extending through the upper finger pad to the central channel of the body of the handle; and
the tip comprising a curved stainless-steel tube with an inner diameter from 3F to 12F, the tip having a free end and a proximal end opposite the free end, the proximal end of the tip being attached to the inlet of the body, the tip having a radius of curvature of 4 to 12 inches and extending for an arc angle of 40 to 60 degrees.
